(19) United States
(12) Patent Application Publication
(10) Pub. No.: US 2011/0082120 A1
(43) Pub. Date: Apr. 7, 2011

(54) SUBSTITUTED THIOACETIC ACID SALICYLATE DERIVATIVES AND THEIR USES

(76) Inventors: Jill C. Milne, Brookline, MA (US); Michael R. Jirousek, Cambridge, MA (US); Jean E. Bemis, Arlington, MA (US); Chi B. Vu, Arlington, MA (US)

(21) Appl. No.: 12/898,453

(22) Filed: Oct. 5, 2010

Related U.S. Application Data
(60) Provisional application No. 61/248,580, filed on Oct. 5, 2009.
(60) Provisional application No. 61/308,721, filed on Feb. 26, 2010.

(51) Int. Cl. A61K 31/609; C07C 233/81; C07C 57/42; A61P 3/00; A61P 9/00; A61P 29/00

(52) U.S. Cl. 514/166; 564/154; 562/431; 514/159

(57) ABSTRACT

The invention relates to substituted thioacetic acid salicylate derivatives; compositions comprising an effective amount of a substituted thioacetic acid salicylate derivative; and methods for treating or preventing an metabolic disease comprising the administration of an effective amount of a substituted thioacetic acid salicylate derivative.

### PRIORITY

This application claim the benefit of U.S. Provisional Application No. 61/248,580, filed Oct. 5, 2009, and U.S. Provisional Application No. 61/308,721, filed Feb. 26, 2010. The entire disclosures of those applications are relied on and incorporated into this application by reference.

### FIELD OF THE INVENTION

The invention relates to substituted thioacetic acid salicylate derivatives, compositions comprising an effective amount of a substituted thioacetic acid salicylate, derivative; and methods for treating or preventing inflammation or a metabolic disease comprising the administration of an effective amount of a substituted thioacetic acid salicylate derivative. All patents, patent applications, and publications cited herein are hereby incorporated by reference in their entireties.

### BACKGROUND OF THE INVENTION

Tetradecylthioacetic Acid (TTA) has been shown to reduce fatty acids and adiposity in animals fed a high fat diet (Wensaas, A. J. et al. Diabetes Obes. Metab. 2009, 11 (11), 1034-1049) and to improve health. Furthermore, in diabetic patients TTA has been shown to shown to improve low-density lipoprotein (“LDL”) levels through lowering Apolipoprotein B (“ApoB”) and raising high density lipoprotein (“HDL”) through increasing Apolipoprotein A1 (“ApoA1”) in the liver (Løvås, K. et al. Diabetes Obes. Metab. 2009, 11 (4), 304-314). TTA has also been shown to reduce inflammation psoriasis (Dyrøy, E. et al. Arterioscler. Thromb. Vasc. Biol. 2005, 25 (7), 1364-1369).

Salicylates have been demonstrated to improve glycemia and reduce fatty acids in patients with type 2 diabetes. Salicylates are thought to achieve this activity through inhibition of the NFκB pathway. Salicylates however can be limited in their therapeutic utility by side effects like tinnitus that occurs at higher doses where optimal efficacy occurs. Salicylates through inhibition of the NFκB pathway also reduce inflammation.

Conjugates of substituted thioacetic acids and polyunsaturated fatty acid analogs that act synergistically with salicylate have improved efficacy and provide safer and more effective therapeutics. These compounds have utility in treating metabolic disease, cardiovascular disease, type 2 diabetes, hypercholesterolemia and dyslipidemia. These compounds may also be used to treat inflammatory conditions such as psoriasis.

The ability to provide the effects of substituted thioacetic acids and salicylates in a synergistic way would provide a great benefit in treating the aforementioned diseases.

### SUMMARY OF THE INVENTION

The invention is based in part on the discovery of substituted thioacetic acid salicylate derivatives and their demonstrated effects in achieving improved treatment that cannot be achieved by administering TTA or unsaturated fatty acids derivatives of TTA alone or in combination with salicylate. These novel compounds are useful in the treatment or prevention of metabolic diseases including atherosclerosis, dyslipidemia, coronary heart disease, hypercholesterolemia, Type 2 diabetes, elevated cholesterol, metabolic syndrome and cardiovascular disease.

Accordingly in one aspect, a molecular conjugate is described which comprises a salicylate and a substituted thioacetic acid covalently linked, and the conjugate is capable of hydrolysis to produce free salicylate and free substituted thioacetic acid.

In another aspect, compounds of Formula I are described:

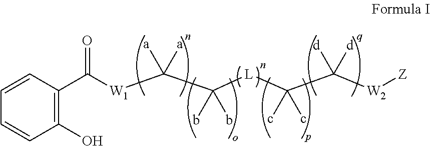

and pharmaceutically acceptable salts, hydrates, solvates, prodrugs, enantiomers, and stereoisomers thereof;

wherein

W₁ and W₂ are each independently null, O, S, NH, NR, or W₁ and W₂ can be taken together can form an imidazolidine or piperazine group;

each a, b, c, and d is independently —H, -D, —CH₃, alkyl —OCH₃, —OCH₂CH₃, —C(O)OR, —O—Z, or benzyl, or two of a, b, c, and d can be taken together, along with the single carbon to which they are bound, to form a cycloalkyl or heterocycle;

each n, o, p, and q is independently 0, 1, or 2;
each L is independently —O—, —S—, —S(O)—, —S(O)₂—, —S—S—, —(C₁-C₆alkyl)-

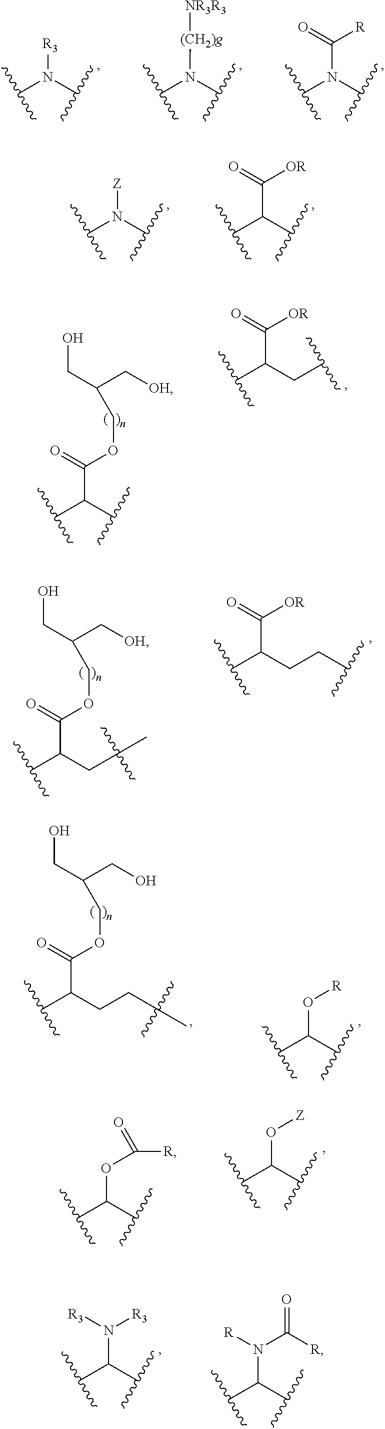

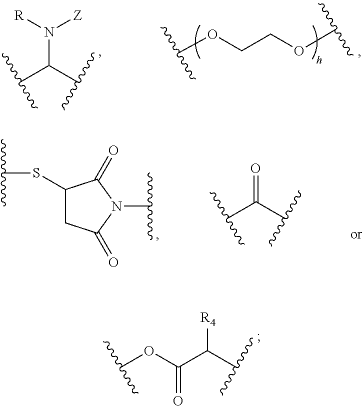

wherein the representation of L is not limited directionally left to right as is depicted, rather either the left side or the right side of L can be bound to the W₁ side of the compound of Formula I;
each g is independently 2, 3 or 4;
each h is independently 1, 2, 3 or 4;
m is 0, 1, 2, or 3; if m is more than 1, then L can be the same or different;

each R₃ is independently H or C₁-C₆ alkyl, or both R₃ groups, when taken together with the nitrogen to which they are attached, can form a heterocycle;

each R₄ is independently e, H or straight or branched C₁-C₁₀ alkyl which can be optionally substituted with OH, NH₂, CO₂R, CONH₂, phenyl, C₆H₄OH, imidazole or arginine;

each e is independently H or any one of the side chains of the naturally occurring amino acids;

each Z is independently —H, or

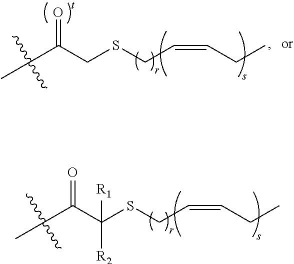

with the proviso that there is at least one

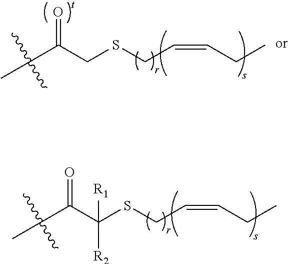

in the compound;

each r is independently 1, 2, 3, 10, 11, 12, 13, 14, 15, or 16,

each s is independently 0, 3, 4, 5, or 6;

with the proviso that if s is 0, then r is 10, 11, 12, 13, 14, 15, or 16;

each t is independently 0 or 1;

R₁ and R₂ are each independently hydrogen, deuterium, —C₁-C₄ alkyl, -halogen, —OH, —C(O)C₁-C₄ alkyl, —O-aryl, —O-benzyl, —OC(O)C₁-C₄ alkyl, —C₁-C₃ alkene, —C₁-C₃ alkyne, —C(O)C₁-C₄ alkyl, —NH₂, —NH(C₁-C₃ alkyl), —N(C₁-C₃ alkyl)₂, —NH(C(O)C₁-C₃ alkyl), —N(C(O)C₁-C₃ alkyl)₂, —SH, —S(C₁-C₃ alkyl), —S(O)C₁-C₃ alkyl, —S(O)₂C₁-C₃ alkyl; and

each R is independently —H, —C(O)—C₁-C₃ alkyl, or straight or branched C₁-C₄ alkyl optionally substituted with OR, NR₂, or halogen;

provided that
when each of m, n, o, p, and q, is 0, W₁ and W₂ are each null, and Z is

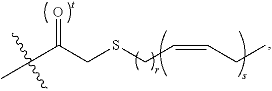

then t must be 0; and
when each of m, n, o, p, and q, is 0, and W₁ and W₂ are each null, then Z must not be

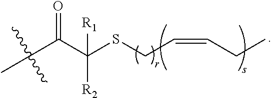

In another aspect, compounds of Formula II are described:

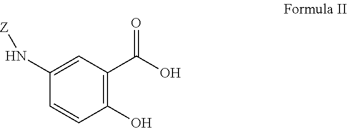

and pharmaceutically acceptable salts, hydrates, solvates, prodrugs, enantiomers, and stereoisomers thereof;

wherein

Z is

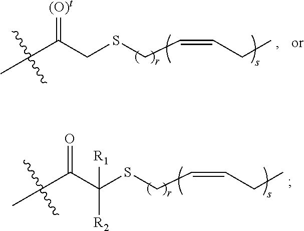

r is 1, 2, 3, 10, 11, 12, 13, 14, 15, or 16,

s is 0, 3, 4, 5, or 6;

with the proviso that if s is 0, then r is 10, 11, 12, 13, 14, 15, or 16;

t is 0 or 1; and

R₁ and R₂ are each independently hydrogen, deuterium, —C₁-C₄ alkyl, -halogen, —OH, —C(O)C₁-C₄ alkyl, —O-aryl, —O-benzyl, —OC(O)C₁-C₄ alkyl, —C₁-C₃ alkene, —C₁-C₃ alkyne, —C(O)C₁-C₄ alkyl, —NH₂, —NH(C₁-C₃ alkyl), —N(C₁-C₃ alkyl)₂, —NH(C(O)C₁-C₃ alkyl), —N(C(O)C₁-C₃ alkyl)₂, —SH, —S(C₁-C₃ alkyl), —S(O)C₁-C₃ alkyl, —S(O)₂C₁-C₃ alkyl.

In Formula I and Formula II, any one or more of H may be substituted with a deuterium. It is also understood in Formula I and Formula II that a methyl substituent can be substituted with a C₁-C₆ alkyl.

Also described are pharmaceutical formulations comprising at least one substituted thioacetic acid salicylate derivatives.

Also described herein are methods of treating a disease susceptible to treatment with a substituted thioacetic acid salicylate derivative in a patient in need thereof by administering to the patient an effective amount of a substituted thioacetic acid salicylate derivative.

Also described herein are methods of treating metabolic diseases by administering to a patient in need thereof an effective amount of a substituted thioacetic acid salicylate derivative.

Also described herein are methods of treating inflammation disorders by administering to a patient in need thereof an effective amount of a substituted thioacetic acid salicylate derivative.

The invention also includes pharmaceutical compositions that comprise an effective amount of a substituted thioacetic acid salicylate derivative and a pharmaceutically acceptable carrier. The compositions are useful for treating or preventing a metabolic disease. The invention includes a substituted thioacetic acid salicylate derivative provided as a pharmaceutically acceptable prodrug, a hydrate, a salt, such as a pharmaceutically acceptable salt, enantiomer, stereoisomer, or mixtures thereof.

The details of the invention are set forth in the accompanying description below. Although methods and materials similar or equivalent to those described herein can be used in the practice or testing of the present invention, illustrative methods and materials are now described. Other features, objects, and advantages of the invention will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this invention belongs. All patents and publications cited in this specification are incorporated herein by reference in their entireties.

### DETAILED DESCRIPTION OF THE INVENTION

Metabolic diseases are a wide variety of medical disorders that interfere with a subject's metabolism. Metabolism is the process a subject's body uses to transform food into energy. Metabolism in a subject with a metabolic disease is disrupted in some way. The substituted thioacetic acid salicylate derivatives possess the ability to treat or prevent metabolic diseases.

The substituted thioacetic acid salicylate derivatives have been designed to bring together substituted thioacetic acid derivatives and a salicylate into a single molecular conjugate. The activity of the substituted thioacetic acid salicylate derivatives is substantially greater than the sum of the individual components of the molecular conjugate, suggesting that the activity induced by the substituted thioacetic acid salicylate derivatives is synergistic.

### DEFINITIONS

The following definitions are used in connection with the substituted thioacetic acid salicylate derivatives:

The term “substituted thioacetic acid salicylate derivatives” includes any and all possible isomers, stereoisomers, enantiomers, diastereomers, tautomers, pharmaceutically acceptable salts, hydrates, solvates, and prodrugs of the fatty substituted thioacetic acid salicylate derivatives described herein.

The articles “a” and “an” are used in this disclosure to refer to one or more than one (i.e., to at least one) of the grammatical object of the article. By way of example, “an element” means one element or more than one element.

The term “and/or” is used in this disclosure to mean either “and” or “or” unless indicated otherwise.

Unless otherwise specifically defined, the term “aryl” refers to cyclic, aromatic hydrocarbon groups that have 1 to 2 aromatic rings, including monocyclic or bicyclic groups such as phenyl, biphenyl or naphthyl. Where containing two aromatic rings (bicyclic, etc.), the aromatic rings of the aryl group may be joined at a single point (e.g., biphenyl), or fused (e.g., naphthyl). The aryl group may be optionally substituted by one or more substituents, e.g., 1 to 5 substituents, at any point of attachment. The substituents can themselves be optionally substituted.

“C₁-C₃ alkyl” refers to a straight or branched chain saturated hydrocarbon containing 1-3 carbon atoms. Examples of a C₁-C₃ alkyl group include, but are not limited to, methyl, ethyl, propyl, and isopropyl.

“C₁-C₄ alkyl” refers to a straight or branched chain saturated hydrocarbon containing 1-4 carbon atoms. Examples of a C₁-C₄ alkyl group include, but are not limited to, methyl, ethyl, propyl, butyl, isopropyl, isobutyl, sec-butyl, and tert-butyl.

“C₁-C₅ alkyl” refers to a straight or branched chain saturated hydrocarbon containing 1-5 carbon atoms. Examples of a C₁-C₅ alkyl group include, but are not limited to, methyl, ethyl, propyl, butyl, pentyl, isopropyl, isobutyl, sec-butyl, and tert-butyl, isopentyl, and neopentyl.

“C₁-C₆ alkyl” refers to a straight or branched chain saturated hydrocarbon containing 1-6 carbon atoms. Examples of a C₁-C₆ alkyl group include, but are not limited to, methyl, ethyl, propyl, butyl, pentyl, hexyl, isopropyl, isobutyl, sec-butyl, tert-butyl, isopentyl, and neopentyl.

The term “cycloalkyl” refers to a cyclic hydrocarbon containing 3-6 carbon atoms. Examples of a cycloalkyl group include, but are not limited to, cyclopropyl, cyclobutyl, cyclopentyl, and cyclohexyl.

It is understood that any of the substitutable hydrogens on an alkyl or cycloalkyl can be substituted with halogen, C₁-C₃ alkyl, hydroxyl, alkoxy, and cyano groups.

The term “heterocycle” as used herein refers to a cyclic hydrocarbon containing 3-6 atoms wherein at least one of the atoms is an O, N, or S. Examples of heterocycles include, but are not limited to, aziridine, oxirane, thiirane, azetidine, oxetane, thietane, pyrrolidine, tetrahydrofuran, tetrahydrothiophene, piperidine, tetrahydropyran, thiane, imidazolidine, oxazolidine, thiazolidine, dioxolane, dithiolane, piperazine, oxazine, dithiane, and dioxane.

The term “any one of the side chains of the naturally occurring amino acids” as used herein means a side chain of any one of the following amino acids: Isoleucine, Alanine, Leucine, Asparagine, Lysine, Aspartate, Methionine, Cysteine, Phenylalanine, Glutamate, Threonine, Glutamine, Tryptophan, Glycine, Valine, Proline, Arginine, Serine, Histidine, and Tyrosine.

The term “substituted thioacetic acid” as used herein means a 2-(alkylthio)acetic acid including polyunsaturated alkyl groups that can mimic the effects of omega-3 fatty acids in vivo. Non-limiting examples of substituted thioacetic acids are 2-(tetradecylthio)acetic acid, 2-((2Z,5Z,8Z,11Z,14Z,17Z)-icosa-2,5,8,11,14,17-hexaenylthio)acetic acid, 2-((2Z,5Z,8Z,11Z,14Z)-heptadeca-2,5,8,11,14-pentaenylthio)acetic acid, and 2-((2Z,5Z,8Z,11Z)-tetradeca-2,5,8,11-tetraenylthio) acetic acid.

The term “salicylic acid” as used herein means the molecule known as salicylic acid and any derivative thereof.

The term “salicylate” as used herein means the esters or salts of salicylic acid and any derivative thereof.

A “subject” is a mammal, e.g., a human, mouse, rat, guinea pig, dog, cat, horse, cow, pig, or non-human primate, such as a monkey, chimpanzee, baboon or rhesus.

The invention also includes pharmaceutical compositions comprising an effective amount of a substituted thioacetic acid salicylate derivative and a pharmaceutically acceptable carrier. The invention includes a Fatty substituted thioacetic acid salicylate derivative provided as a pharmaceutically acceptable prodrug, hydrate, salt, such as a pharmaceutically acceptable salt, enantiomers, stereoisomers, or mixtures thereof.

Representative “pharmaceutically acceptable salts” include, e.g., water-soluble and water-insoluble salts, such as the acetate, amsonate (4,4-diaminostilbene-2,2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fiunarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, magnesium, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosalicylate, suramate, tannate, tartrate, teoclate, tosylate, triethiodide, and valerate salts.

The term “metabolic disease” as used herein refers to disorders, diseases and syndromes involving dyslipidemia, and the terms metabolic disorder, metabolic disease and metabolic syndrome are used interchangeably herein.

An “effective amount” when used in connection with a substituted thioacetic acid salicylate derivative is an amount effective for treating or preventing a metabolic disease.

The term “carrier”, as used in this disclosure, encompasses carriers, excipients, and diluents and means a material, composition or vehicle, such as a liquid or solid filler, diluent, excipient, solvent or encapsulating material, involved in carrying or transporting a pharmaceutical agent from one organ, or portion of the body, to another organ, or portion of the body.

The term “treating”, with regard to a subject, refers to improving at least one symptom of the subject's disorder. Treating can be curing, improving, or at least partially ameliorating the disorder.

The term “disorder” is used in this disclosure to mean, and is used interchangeably with, the terms disease, condition, or illness, unless otherwise indicated.

The term “administer”, “administering”, or “administration” as used in this disclosure refers to either directly administering a compound or pharmaceutically acceptable salt of the compound or a composition to a subject, or administering a prodrug derivative or analog of the compound or pharmaceutically acceptable salt of the compound or composition to the subject, which can form an equivalent amount of active compound within the subject's body.

The term “prodrug,” as used in this disclosure, means a compound which is convertible in vivo by metabolic means (e.g., by hydrolysis) to a substituted thioacetic acid salicylate derivative.

The following abbreviations are used herein and have the indicated definitions: Boc and BOC are tert-butoxycarbonyl, Boc₂O is di-tert-butyl dicarbonate, CDI is 1,1′-carbonyldiimidazole, DCC is N,N′-dicyclohexylcarbodiimide, DIEA is N,N-diisopropylethylamine, dimethoxyethane is 1,2-dimethoxyethane, DMAP is 4-dimethylaminopyridine, DOSS is sodium dioctyl sulfosuccinate, EDC and EDCI are 1-ethyl-3-(3-dimethylaminopropyl)carbodiimide hydrochloride, EtOAc is ethyl acetate, GAPDH is glyceraldehyde 3-phosphate dehydrogenase, h is hour, HATU is 2-(7-aza-1H-benzotriazole-1-yl)-1,1,3,3-tetramethyluronium hexafluorophosphate, HIV is human immunodeficiency virus, HPMC is hydroxypropyl methylcellulose, MCP is monocyte chemotactic protein, oxone is potassium peroxymonosulfate, PBMC is peripheral blood mononuclear cell, PBS is phosphate buffered saline, PCR is polymerase chain reaction, Pd/C is palladium on carbon, RNA is ribonucleic acid, RT is room temperature, TFA is trifluoroacetic acid, TGPS is tocopherol propylene glycol succinate, THF is tetrahydrofuran, TNF is tumor necrosis factor, and VCAM is vascular cell adhesion molecule.

Accordingly in one aspect, a molecular conjugate is described which comprises a salicylate and a substituted thioacetic acid covalently linked, and the conjugate is capable of hydrolysis to produce free salicylate and free substituted thioacetic acid. In some embodiments, the hydrolysis is enzymatic.

In another aspect, the present invention provides substituted thioacetic acid salicylate derivatives according to Formula I:

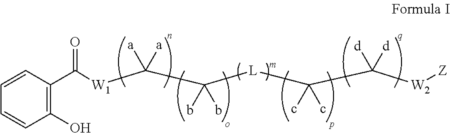

and pharmaceutically acceptable salts, hydrates, solvates, prodrugs, enantiomers, and stereoisomers thereof;

wherein

R₁, R₂, R₃, R₄, R, W₁, W₂, L, Z, a, c, b, d, e, g, h, m, n, o, p, q, r, s, and t are as defined above for Formula I,

with the proviso that there is at least one

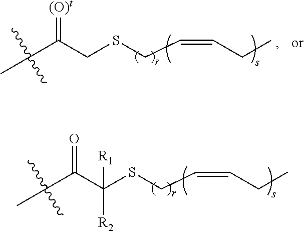

in the compound.

In some embodiments, W₁ is O.

In some embodiments, W₂ is O.

In some embodiments, W₁ is NH.

In some embodiments, W₂ is NH.

In some embodiments, a and c are each independently H, or CH₃.

In some embodiments, m is 0.

In some embodiments, each L is independently —S—, —S(O)—, —S(O)₂—, or —S—S—.

In some embodiments, each L is independently —O—,

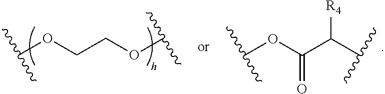

In some embodiments, each L is independently

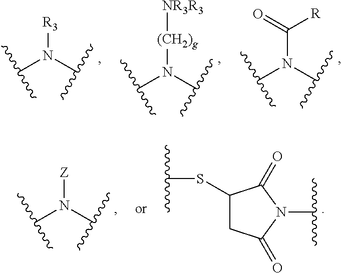

In some embodiments, each L is independently

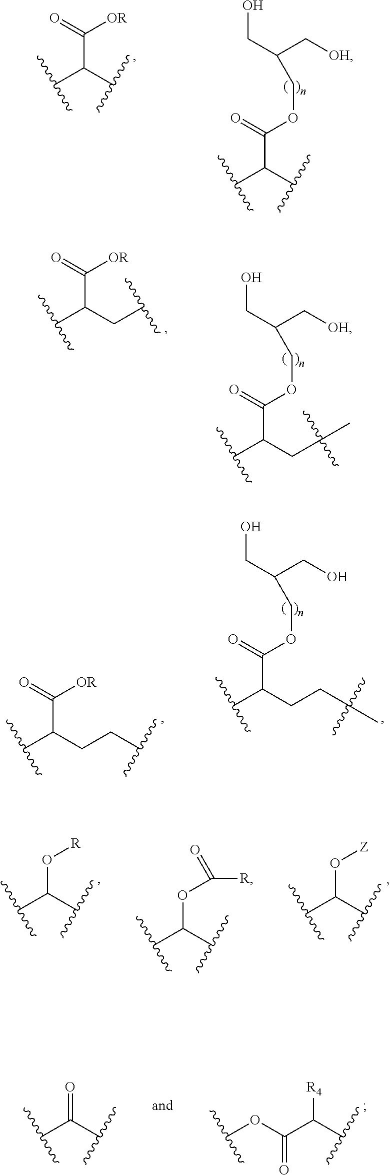

In some embodiments, each L is independently

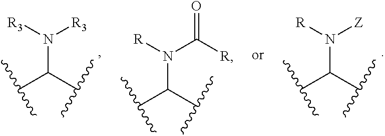

In some embodiments, one b is O—Z, Z is

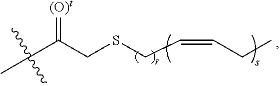

and t is 1.

In some embodiments, d is C(O)OR.

In some embodiments n, o, p, and q are each 1.

In some embodiments, two of n, o, p, and q are each 1.

In other embodiments, three of n, o, p, and q are each 1.

In some embodiments, t is 1.

In another aspect, the present invention provides substituted thioacetic acid salicylate derivatives according to Formula II:

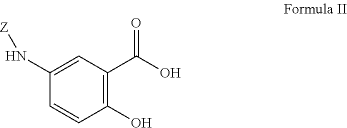

wherein

Z, r, s, and t are as defined above for Formula II.

In some embodiments, t is 1.

The following embodiments are descriptive of Formula I and Formula II.

In some embodiments, Z is

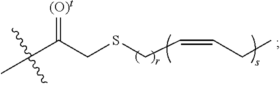

and r is 1.

In some embodiments, Z is

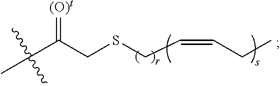

and r is 2.

In some embodiments, Z is

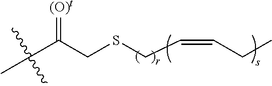

and r is 3.

In some embodiments, Z is

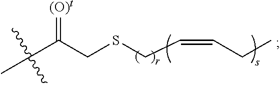

and r is 13, s is 0 and t is 1

In some embodiments, Z is

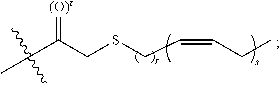

and r is 14, s is 0 and t is 1.

In some embodiments, Z is

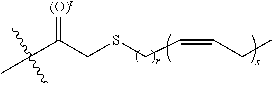

and s is 5.

In some embodiments, Z is

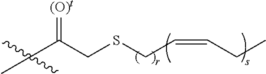

and s is 6.

In some embodiments, Z is

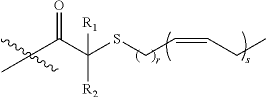

and r is 1.

In other embodiments, Z is

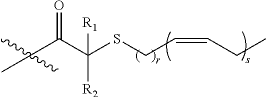

and r is 2.

In some embodiments, Z is

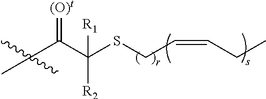

and r is 3.

In other embodiments, Z is

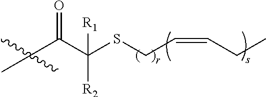

and r is 14 and s is 0.

In some embodiments, Z is

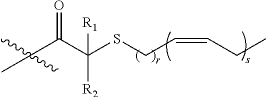

and s is 4.

In some embodiments, Z is

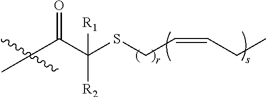

and s is 5.

In other embodiments, Z is

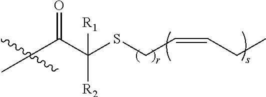

and s is 6.

In other illustrative embodiments, compounds of Formula I and Formula II are as set forth below:
2-hydroxy-N-(2-(2-(2-((2Z,5Z,8Z,11Z,14Z,17Z)-icosa-2,5,8,11,14,17-hexaenylthio)acetamido)ethoxy)ethyl)benzamide (I-1),
2-hydroxy-N-(2-((2-(2-((2Z,5Z,8Z,11Z,14Z,17Z)-icosa-2,5,8,11,14,17-hexaenylthio)acetamido)ethyl)(methyl)amino)ethyl)benzamide (I-2),
2-hydroxy-N-(2-(2-(2-(2-((2Z,5Z,8Z,11Z,14Z,17Z)-icosa-2,5,8,11,14,17-hexaenylthio)acetamido)ethyl)disulfanyl)ethyl)benzamide (I-3),
2-hydroxy-N-(2-(1-(2-(2-((2Z,5Z,8Z,11Z,14Z,17Z)-icosa-2,5,8,11,14,17-hexaenylthio)acetamido)ethyl)-2,5-dioxopyrrolidin-3-ylthio)ethyl)benzamide (I-4),
3-(2-hydroxybenzamido)-4-methoxy-4-oxobutan-2-yl 2-(2-((2Z,5Z,8Z,11Z,14Z,17Z)-icosa-2,5,8,11,14,17-hexaen-1-ylthio)acetamido)-3-methylbutanoate (I-5),
1,3-dihydroxypropan-2-yl 2-(2-hydroxybenzamido)-6-(2-((2Z,5Z,8Z,11Z,14Z,17Z)-icosa-2,5,8,11,14,17-hexaenylthio)acetamido)hexanoate (I-6),
and
2-hydroxy-5-(2-(2-(2-(2-((2Z,5Z,8Z,11Z,14Z,17Z)-icosa-2,5,8,11,14,17-hexaenylthio)acetamido)ethyl)disulfanyl)acetamido)benzoic acid (I-7).

### Methods for Using Substituted Thioacetic Acid Salicylate Derivatives

The invention also includes methods for treating metabolic diseases such as the treatment or prevention of metabolic diseases including atherosclerosis, dyslipidemia, coronary heart disease, hypercholesterolemia, Type 2 diabetes, elevated cholesterol, metabolic syndrome and cardiovascular disease.

The invention also includes methods for treating inflammatory disease such as psoriasis.

In one embodiment, the method comprises contacting a cell with a substituted thioacetic acid salicylate derivatives in an amount sufficient to decrease the release of triglycerides or VLDL or LDL or cause an increase in reverse cholesterol transport or increase HDL concentrations.

Also provided in the invention is a method for inhibiting, preventing, or treating a metabolic disease, or symptoms of a metabolic disease, in a subject. Examples of such disorders include, but are not limited to atherosclerosis, dyslipidemia, hypertriglyceridemia, hypertension, heart failure, cardiac arrhythmias, low HDL levels, high LDL levels, sudden death, stable angina, coronary heart disease, acute myocardial infarction, secondary prevention of myocardial infarction, cardiomyopathy, endocarditis, type 2 diabetes, insulin resistance, impaired glucose tolerance, hypercholesterolemia, stroke, hyperlipidemia, hyperlipoproteinemia, chronic kidney disease, intermittent claudication, hyperphosphatemia, carotid atherosclerosis, peripheral arterial disease, diabetic nephropathy, hypercholesterolemia in HIV infection, acute coronary syndrome (ACS), non-alcoholic fatty liver disease, arterial occlusive diseases, cerebral arteriosclerosis, cerebrovascular disorders, myocardial ischemia and diabetic autonomic neuropathy.

In some embodiments, the subject is administered an effective amount of a substituted thioacetic acid salicylate derivative.

The invention also includes pharmaceutical compositions useful for treating or preventing a metabolic disease, or for inhibiting a metabolic disease, or more than one of these activities. The compositions can be suitable for internal use and comprise an effective amount of a substituted thioacetic acid salicylate derivative and a pharmaceutically acceptable carrier. The substituted thioacetic acid salicylate derivatives are especially useful in that they demonstrate very low peripheral toxicity or no peripheral toxicity.

The substituted thioacetic acid salicylate derivatives can each be administered in amounts that are sufficient to treat or prevent a metabolic disease or prevent the development thereof in subjects.

Administration of the substituted thioacetic acid salicylate derivatives can be accomplished via any mode of administration for therapeutic agents. These modes include systemic or local administration such as oral, nasal, parenteral, transdermal, subcutaneous, vaginal, buccal, rectal or topical administration modes.

Depending on the intended mode of administration, the compositions can be in solid, semi-solid or liquid dosage form, such as, for example, injectables, tablets, suppositories, pills, time-release capsules, elixirs, tinctures, emulsions, syrups, powders, liquids, suspensions, or the like, sometimes in unit dosages and consistent with conventional pharmaceutical practices. Likewise, they can also be administered in intravenous (both bolus and infusion), intraperitoneal, subcutaneous or intramuscular form, all using forms well known to those skilled in the pharmaceutical arts.

Illustrative pharmaceutical compositions are tablets and gelatin capsules comprising a substituted thioacetic acid salicylate derivative and a pharmaceutically acceptable carrier, such as: a) a diluent, e.g., purified water, triglyceride oils, such as hydrogenated or partially hydrogenated vegetable oil, or mixtures thereof, corn oil, olive oil, sunflower oil, safflower oil, fish oils, such as EPA or DHA, or their esters or triglycerides or mixtures thereof, omega-3 fatty acids or derivatives thereof, lactose, dextrose, sucrose, mannitol, sorbitol, cellulose, sodium, saccharin, glucose and/or glycine; b) a lubricant, e.g., silica, talcum, stearic acid, its magnesium or calcium salt, sodium oleate, sodium stearate, magnesium stearate, sodium benzoate, sodium acetate, sodium chloride and/or polyethylene glycol; for tablets also; c) a binder, e.g., magnesium aluminum silicate, starch paste, gelatin, tragacanth, methylcellulose, sodium carboxymethylcellulose, magnesium carbonate, natural sugars such as glucose or beta-lactose, corn sweeteners, natural and synthetic gums such as acacia, tragacanth or sodium alginate, waxes and/or polyvinylpyrrolidone, if desired; d) a disintegrant, e.g., starches, agar, methyl cellulose, bentonite, xanthan gum, alginic acid or its sodium salt, or effervescent mixtures; e) absorbent, colorant, flavorant, and sweetener; f) an emulsifier or dispersing agent, such as Tween 80, Labrasol, HPMC, DOSS, caproyl 909, labrafac, labrafil, peceol, transcutol, capmul MCM, capmul PG-12, captex 355, gelucire, vitamin E TGPS or other acceptable emulsifier; and/or g) an agent that enhances absorption of the compound such as cyclodextrin, hydroxypropyl—cyclodextrin, PEG400, PEG200.

Liquid, particularly injectable, compositions can, for example, be prepared by dissolution, dispersion, etc. For example, the substituted thioacetic acid salicylate derivative is dissolved in or mixed with a pharmaceutically acceptable solvent such as, for example, water, saline, aqueous dextrose, glycerol, ethanol, and the like, to thereby form an injectable isotonic solution or suspension. Proteins such as albumin, chylomicron particles, or serum proteins can be used to solubilize the substituted thioacetic acid salicylate derivatives.

The substituted thioacetic acid salicylate derivatives can be also formulated as a suppository that can be prepared from fatty emulsions or suspensions; using polyalkylene glycols such as propylene glycol, as the carrier.

The substituted thioacetic acid salicylate derivatives can also be administered in the form of liposome delivery systems, such as small unilamellar vesicles, large unilamellar vesicles, and multilamellar vesicles. Liposomes can be formed from a variety of phospholipids, containing cholesterol, stearylamine or phosphatidylcholines. In some embodiments, a film of lipid components is hydrated with an aqueous solution of drug to a form lipid layer encapsulating the drug, as described in U.S. Pat. No. 5,262,564, the contents of which are herein incorporated by reference in their entirety.

Substituted thioacetic acid salicylate derivatives can also be delivered by the use of monoclonal antibodies as individual carriers to which the substituted thioacetic acid salicylate derivatives are coupled. The substituted thioacetic acid salicylate derivatives can also be coupled with soluble polymers as targetable drug carriers. Such polymers can include polyvinylpyrrolidone, pyran copolymer, polyhydroxypropylmethacrylamide-phenol, polyhydroxyethylaspanamidephenol, or polyethyleneoxidepolylysine substituted with palmitoyl residues. Furthermore, the substituted thioacetic acid salicylate derivatives can be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates, and cross-linked or amphipathic block copolymers of hydrogels. In one embodiment, substituted thioacetic acid salicylate derivatives are not covalently bound to a polymer, e.g., a polycarboxylic acid polymer, or a polyacrylate.

Parenteral injectable administration is generally used for subcutaneous, intramuscular or intravenous injections and infusions. Injectables can be prepared in conventional forms, either as liquid solutions or suspensions or solid forms suitable for dissolving in liquid prior to injection.

Compositions can be prepared according to conventional mixing, granulating or coating methods, respectively, and the present pharmaceutical compositions can contain from about 0.1% to about 80%, from about 5% to about 60%, or from about 1% to about 20% of the substituted thioacetic acid salicylate derivative by weight or volume.

The dosage regimen utilizing the substituted thioacetic acid salicylate derivative is selected in accordance with a variety of factors including type, species, age, weight, sex, and medical condition of the patient; the severity of the condition to be treated; the route of administration; the renal or hepatic function of the patient; and the particular substituted thioacetic acid salicylate derivative employed. A physician or veterinarian of ordinary skill in the art can readily determine and prescribe the effective amount of the drug required to prevent, counter or arrest the progress of the condition.

Effective dosage amounts of the present invention, when used for the indicated effects, range from about 20 mg to about 2,000 mg of the substituted thioacetic acid salicylate derivative per day. Compositions for in vivo or in vitro use can contain about 50, 75, 100, 150, 250, 350, 500, 750, 1,000, 1,250, 1,500, 2,000, or 2,500 mg of the substituted thioacetic acid salicylate derivative. In one embodiment, the compositions are in the form of a tablet that can be scored. Effective plasma levels of the substituted thioacetic acid salicylate derivative can range from about 0.2 mg to about 200 mg/kg of body weight per day. Appropriate dosages of the substituted thioacetic acid salicylate derivatives can be determined as set forth in Goodman, L. S.; Gilman, A. The Pharmacological Basis of Therapeutics, 5th ed.; MacMillan: New York, 1975, pp. 201-226.

Substituted thioacetic acid salicylate derivatives can be administered in a single daily dose, or the total daily dosage can be administered in divided doses of two, three or four times daily. Furthermore, substituted thioacetic acid salicylate derivatives can be administered in intranasal form via topical use of suitable intranasal vehicles, or via transdermal routes, using those forms of transdermal skin patches well known to those of ordinary skill in that art. To be administered in the form of a transdermal delivery system, the dosage administration can be continuous rather than intermittent throughout the dosage regimen. Other illustrative topical preparations include creams, ointments, lotions, aerosol sprays, and gels, wherein the concentration of the substituted thioacetic acid salicylate derivative ranges from about 0.1% to about 15%, w/w or w/v.

### Methods of Making

### Methods for Making the Substituted Thioacetic Acid Salicylate Derivatives

Examples of synthetic pathways useful for making substituted thioacetic acid salicylate derivatives of Formula I and Formula II are set forth in the Examples below and generalized in Schemes 1-10.

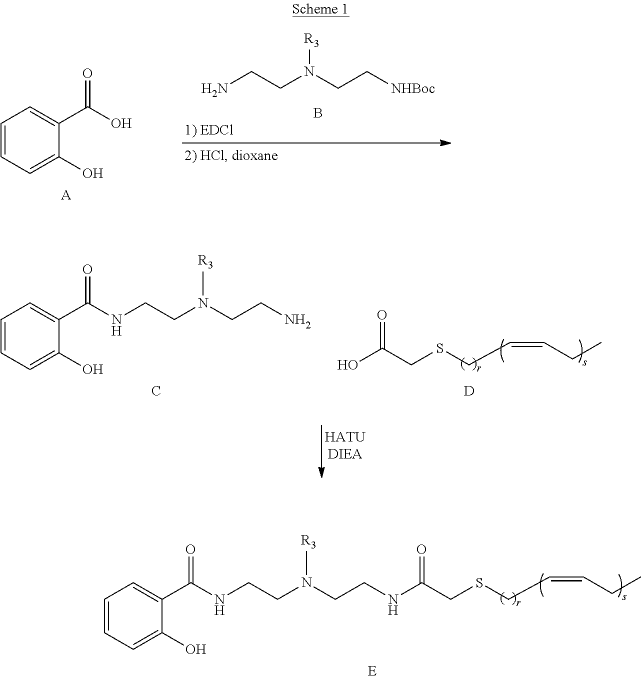

wherein R₃, r, and s are as defined above.

The mono-BOC protected amine of the formula B can be obtained from commercial sources or prepared according to the procedures outlined in Krapcho et al. Synthetic Commun. 1990, 20, 2559-2564. Compound A can be amidated with the amine B using a coupling reagent such as DCC, CDI, EDC, or optionally with a tertiary amine base and/or catalyst, e.g., DMAP, followed by deprotection of the BOC group with acids such as TFA or HCl in a solvent such as CH₂Cl₂ or dioxane to produce the coupled compound C. Activation of compound C with a coupling agent such as HATU in the presence of an amine such as DIEA followed by addition of a fatty acid of formula D affords compounds of the formula E.

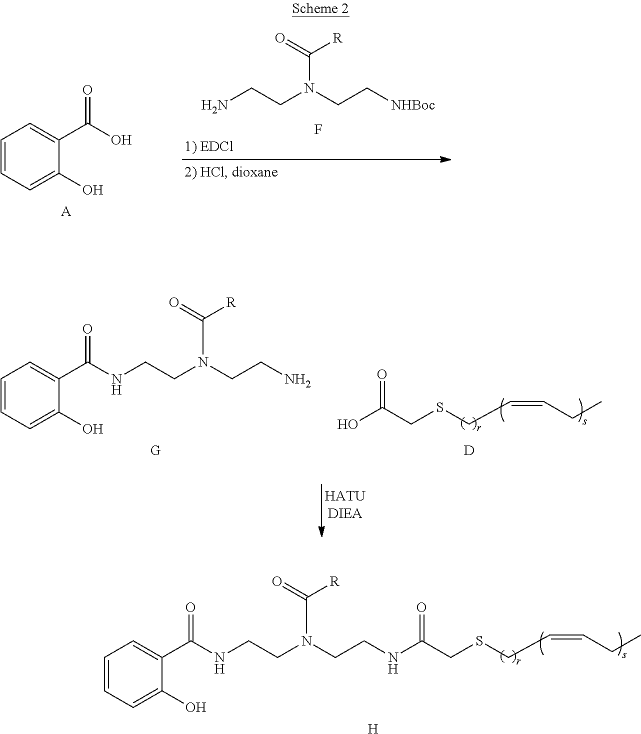

wherein R, r, and s are as defined above.

The acylated amine of the formula F can be prepared using the procedures outlined in Andruszkiewicz et al. Synthetic Commun. 2008, 38, 905-913. Compound A can be amidated with the amine F using a coupling reagent such as DCC, CDI, EDC, or optionally with a tertiary amine base and/or catalyst, e.g., DMAP, followed by deprotection of the BOC group with acids such as TFA or HCl in a solvent such as CH₂Cl₂ or dioxane to produce the coupled compound G. Activation of compound G with a coupling agent such as HATU in the presence of an amine such as DIEA followed by addition of a fatty acid of formula D affords compounds of the formula H.

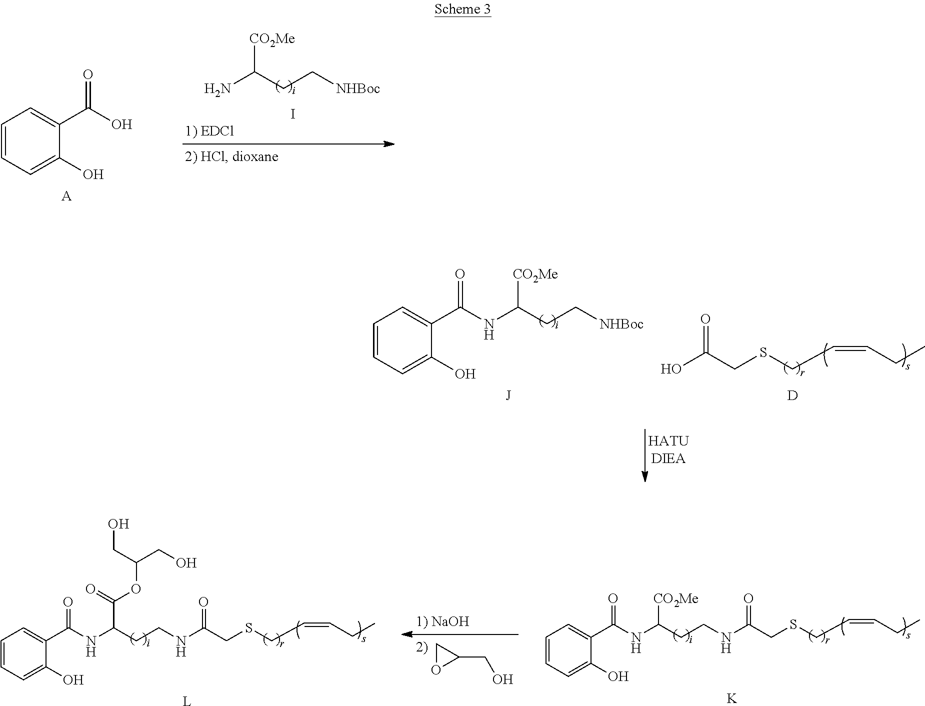

wherein r and s are as defined above.

Compound A can be amidated with the corresponding amine I (where i=0, 1, 2 or 3) using a coupling reagent such as DCC, CDI, EDC, or optionally with a tertiary amine base and/or catalyst, e.g., DMAP, followed by deprotection of the BOC group with acids such as TFA or HCl in a solvent such as CH₂Cl₂ or dioxane to produce the coupled compound J. Activation of compound J with a coupling agent such as HATU in the presence of an amine such as DIEA followed by addition of a fatty acid of formula D affords compounds of the formula K. Hydrolysis of the ester under basic conditions such as NaOH or LiOH produces the corresponding acid, which can be coupled with glycidol to afford compounds of the formula L.

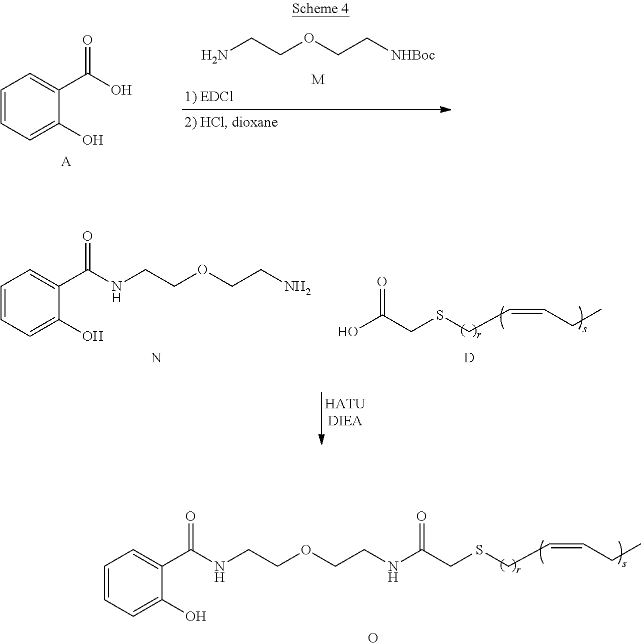

wherein r and s are as defined above.

The amine M can be prepared according to the procedures outlined in Dahan et al. J. Org. Chem. 2007, 72, 2289-2296. Compound A can be coupled with the amine M using a coupling reagent such as DCC, CDI, EDC, or optionally with a tertiary amine base and/or catalyst, e.g., DMAP, followed by deprotection of the BOC group with acids such as TFA or HCl in a solvent such as CH₂Cl₂ or dioxane to produce the coupled compound N. Activation of compound N with a coupling agent such as HATU in the presence of an amine such as DIEA followed by addition of a fatty acid of formula D affords compounds of the formula O.

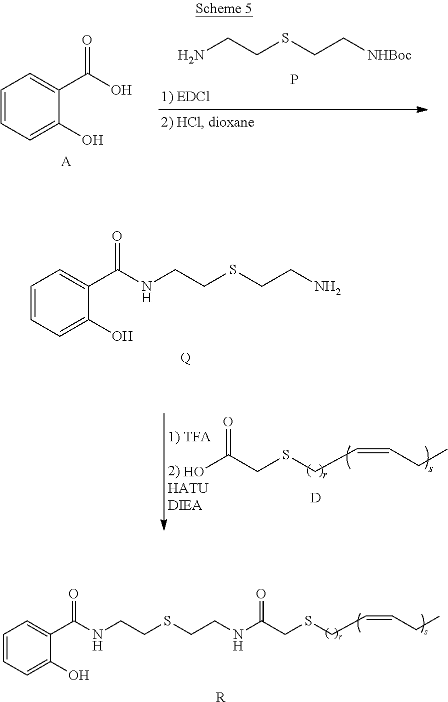

wherein r and s are as defined above.

Compound A can be amidated with the commercially available amine P using a coupling reagent such as DCC, CDI, EDC, or optionally with a tertiary amine base and/or catalyst, e.g., DMAP, to afford compound Q. The BOC group in compound Q can be removed with acids such as TFA or HCl in a solvent such as CH₂Cl₂ or dioxane and the resulting amine can be coupled with a fatty acid of formula D using a coupling agent such as HATU in the presence of an amine such as DIEA to afford compounds of the formula R. To those skilled in the art, the sulfur group in formula Q can be oxidized to the corresponding sulfoxide or sulfone using an oxidizing agent such as H₂O₂ or oxone.

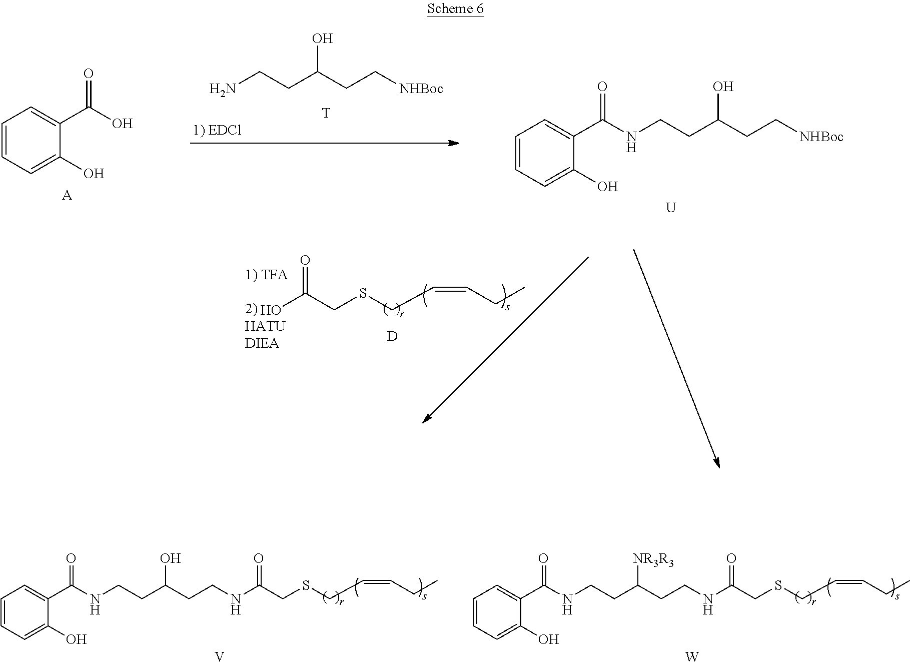

wherein R₃, r, and s are as defined above.

The amine T can be prepared from the commercially available diamine according to the procedures outlined in Dahan et al. J. Org. Chem. 2007, 72, 2289-2296. Compound A can be amidated with the amine T using a coupling reagent such as DCC, CDI, EDC, or optionally with a tertiary amine base and/or catalyst, e.g., DMAP, to afford compound U. The BOC group of compound U can be removed with acids such as TFA or HCl in a solvent such as CH₂Cl₂ or dioxane and the resulting amine can be coupled with a fatty acid of formula D using HATU in the presence of an amine such as DIEA to afford compounds of the formula V. To those skilled in the art, the hydroxyl group in compound U can be further acylated or converted to an amino group by standard mesylation chemistry followed by displacement with sodium azide and hydrogenation over a catalyst such as Pd/C. The amine can be further acylated or alkylated, followed by the removal of the BOC group. The resulting amine can be coupled with a fatty acid of the formula D to afford compounds of the formula W.

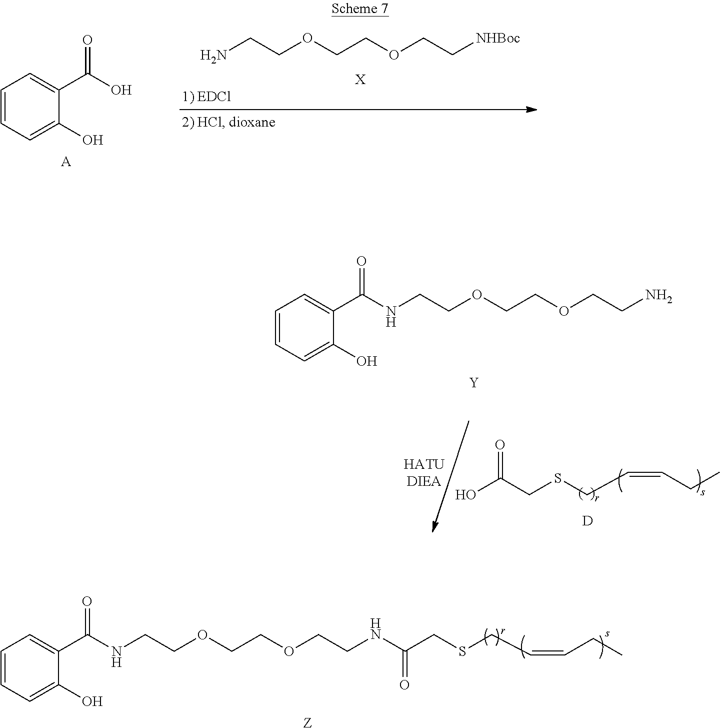

wherein r and s are as defined above.

Compound A can be amidated with the commercially available amine X using a coupling reagent such as DCC, CDI, EDC, optionally with a tertiary amine base and/or catalyst, e.g., DMAP to afford compound Y. The BOC group in compound Y can be removed with acids such as TFA or HCl in a solvent such as CH₂Cl₂ or dioxane. The resulting amine can be coupled with a fatty acid of the formula D using a coupling agent such as HATU in the presence of an amine such as DIEA to afford compounds of the formula Z.

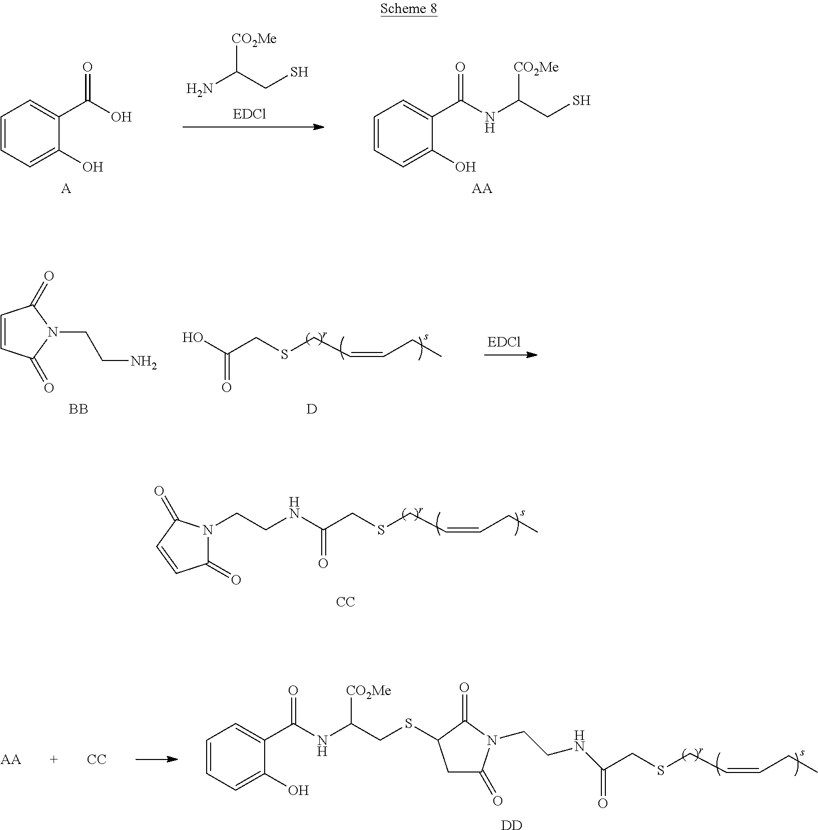

wherein r and s are as defined above.

Compound A can be amidated with the commercially available cysteine methyl ester using a coupling reagent such as DCC, CDI, EDC, or optionally with a tertiary amine base and/or catalyst, e.g., DMAP, to afford compound AA. The commercially available maleimide derivative BB can be coupled with a fatty acid of the formula D using a coupling agent such as HATU or EDCI to afford compounds of the formula CC. Compound AA can be coupled to compounds of the formula CC in a solvent such as acetonitrile to afford compounds of the formula DD.

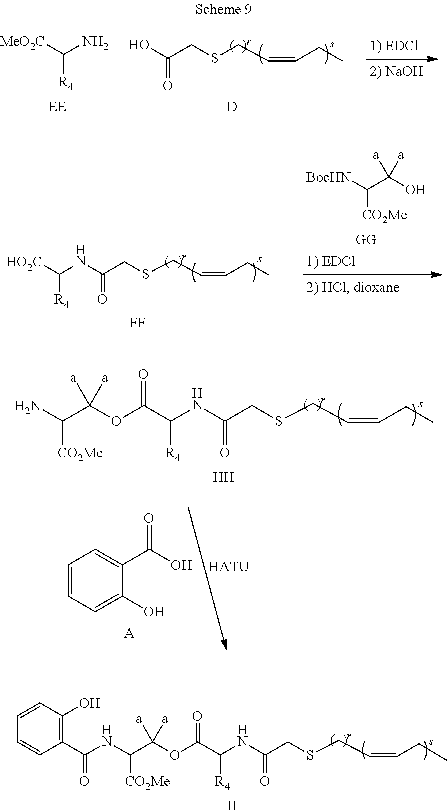

wherein R₄, a, r, and s are as defined above.

The commercially available amino acid esters EE can be coupled with a fatty acid of the formula D using a coupling agent such as EDCI or HATU, followed by alkaline hydrolysis of the methyl ester to afford compounds of the formula FF. Compounds of the formula FF can be coupled with the commercially available BOC-amino acid derivatives GG using a coupling agent such as EDCI or HATU. The BOC group can be removed by treatment with acids such as TFA or HCl to afford compounds of the formula HH which can then be coupled with compound A to afford compounds of the formula II.

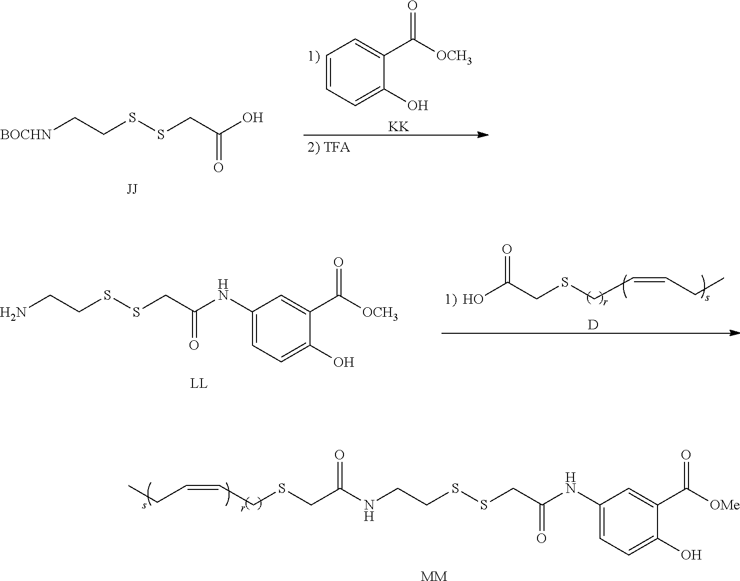

wherein r and s are as defined above.

The acid JJ can be prepared using literature procedures (Méry, J. et al. Peptide Res. 1992, 5 (4), 233-240). Compound JJ can be coupled with aniline KK using a suitable coupling agent such as DCC, CDI, EDC, or optionally with a tertiary amine base and/or catalyst, e.g., DMAP, which, after deprotection with an acid such as TFA or HCl provides Compound LL. Compound LL can be coupled with a fatty acid of the formula D using a suitable coupling agent such as HATU, CDI, EDC, or optionally with a tertiary amine base and/or catalyst, e.g., DMAP, to produce compounds of the formula MM. Compounds MM can be hydrolyzed to the free benzoic acid analogs using standard basic saponification methods such as NaOH or LiOH.

### EXAMPLES

The disclosure is further illustrated by the following examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

### Example 1

### Effect of Substituted Thioacetic Acid Salicylate Derivatives on Inflammatory Markers in Endothelial Cells

Methods have been described to measure TNFα activated endothelial cells and TTA has been shown to block VCAM-1, IL-8 expression (Dyrøy, E. et al. Arterioscler. Thromb. Vasc. Biol. 2005, 25 (7), 1364-1369).

### Cell Experiments

Human Umbilical Vein Endothelial Cells (HUVEC) were purchased from PromoCell (C-12250; Heidelberg, Germany) and grown in Endothelial Cell Growth Medium (C-22010; PromoCell). HUVEC, which was used between passages 1 and 4, were maintained in medium in a humidified chamber containing 5% CO₂ at 37° C. Cells were cultured in 25 cm² or 75 cm² culture flasks (Sarsted, Nümbrecht, Germany) near confluence and the medium was exchanged every 48 hours. When studying endothelial cell activation and leukocyte adhesion, HUVEC of confluent cultures were trypsinized (0.05% wt/vol trypsin and 5 mmol/L EDTA containing Ca²⁺ free solution) and seeded at a density of 20,000 cells/well. At day zero, different concentrations of TTA and after 72 hours of TTA exposure, different concentrations of human recombinant tumor necrosis factor alpha (TNFα) (Sigma; dissolved in PBS) and PBS were added to the culture medium. Six and 20 hours thereafter, cell pellets and cell-free supernatants were harvested and stored at −80° C. To examine adhesion of monocytes and neutrophils to HUVEC, confluent HUVEC monolayers were grown in 96-well tissue culture plates (Becton Dickinson Labware, San José, Calif.). After 72 hours of TTA exposure, half of the wells were stimulated with TNFα (10 ng/mL) for 24 hours to obtain activated endothelial cells. After stimulation and washing, freshly isolated monocytes and granulocytes from healthy human controls were added (1.5×10⁵/well) and were allowed to attach for 30 minutes at 37° C. Thereafter, non-adherent cells were removed by gently aspiration, and the wells were washed twice with warm PBS. Adherent cells were fixed in 4% paraformaldehyde for 30 minutes and counted in four separate high-power fields in each well by phase contrast microscopy (Nikon Phase Contrast-2, Tokyo, Japan). Cell proliferation was assessed by ^{[3H]}Thymidine incorporation as determined by liquid scintillation counting after 4 hours of incubation with 1.0 μCi/well. The endotoxin levels of all stimulants and culture media were less than 10 pg/mL (Limulus Amebocyte Assay; BioWhittaker, Walkersville, Md.).

### Quantitative Real-Time RT-PCR

Total RNA was isolated from HUVEC, mouse liver and PBMC using RNeasy Minikit (Qiagen, Hilden, Germany), and reversed-transcribed using a reverse transcriptase kit (Applied Biosystems, Foster City, Calif.). Sequence-specific primers and TaqMan probes were designed using the Primer Express software version 1.5 (Applied Biosystems). Quantification of mRNA was performed using the ABI Prism7000 (Applied Biosystems). The housekeeping genes β-actin, GAPDH and 18S RNA were included as endogenous normalization controls to adjust for unequal amounts of RNA. Similar patterns were obtained whether normalized to β-actin, GAPDH or 18S RNA.

### Enzyme Immunoassays (EIAs)

IL-8, MCP-1, soluble (s) VCAM-1 and TNFα protein levels were measured by EIAs (R&D Systems, Minneapolis, Minn.).

### Compounds

The following non-limiting compound examples serve to illustrate further embodiments of the substituted thioacetic acid salicylate derivatives. It is to be understood that any embodiments listed in the Examples section are embodiments of the substituted thioacetic acid salicylate derivatives and, as such, are suitable for use in the methods and compositions described above.

### Example 2

### Preparation of 2-hydroxy-5-(2-(2-(2-(2-((2Z,5Z,8Z,11Z,14Z,17Z)-eicosa-2,5,8,11,14,17-hexaenylthio)acetamido)ethyl)disulfanyl)acetamido)benzoic acid (I-7)

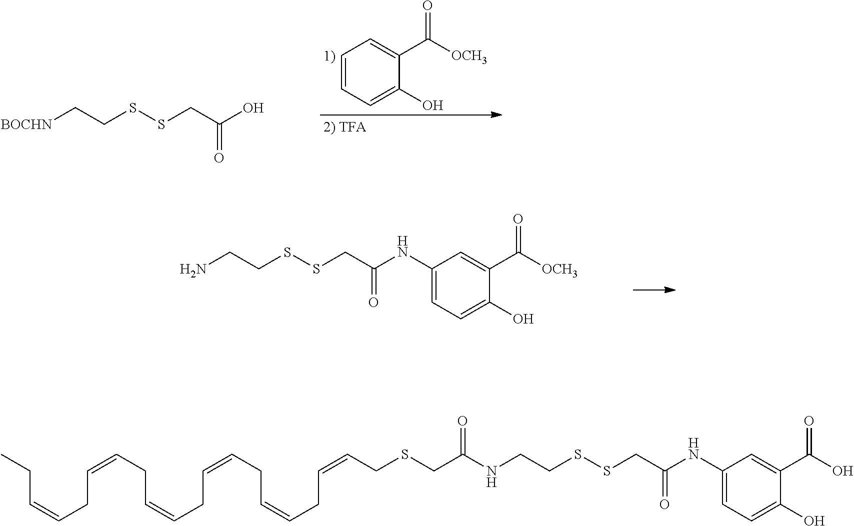

2-(2-(2-(tert-butoxycarbonyl)ethyl)disulfanyl)acetic acid (1 mmol), which is synthesized according to the procedure outlined in Méry, J. et al. Peptide Res. 1992, 5 (4), 233-240, is dissolved in CH₂Cl₂ and to this is added EDCI (1.3 mmol) and methyl 5-amino-2-hydroxybenzoate (1 mmol). The reaction is stirred (RT, 4 h) and then partitioned between CH₂Cl₂ and water. The aqueous layer is extracted with CH₂Cl₂ and the combined organic extracts are washed with water, brine and dried over MgSO₄. Solvent evaporation and purification by silica gel chromatography affords methyl 5-(2-(2-(2-tert-butoxycarbonylaminoethyl)disulfanyl)acetamido)-2-hydroxybenzoate.

Methyl 5-(2-(2-(2-tert-butoxycarbonylaminoethyl)disulfanyl)acetamido)-2-hydroxybenzoate is dissolved in CH₂Cl₂ and TFA and stirred (RT, 4 h). Solvent evaporation affords methyl 5-(2-(2-(2-aminoethyl)disulfanyl)acetamido)-2-hydroxybenzoate, which is then added to a solution of 2Z,5Z,8Z,11Z,14Z,17Z-eicosa-2,5,8,11,14,17-hexaenylthioacetic acid and EDCI. The mixture is stirred (RT, 4 h) and then partitioned between CH₂Cl₂ and water. The aqueous layer is extracted with CH₂Cl₂ and the combined organic extracts are washed with water, brine and dried over MgSO₄. Solvent evaporation and purification by silica gel chromatography affords methyl 2-hydroxy-5-(2-(2-(2-(2-((2Z,5Z,8Z,11Z,14Z,17Z)-eicosa-2,5,8,11,14,17-hexaenylthio)acetamido)ethyl)disulfanyl)acetamido)benzoate.

Methyl 2-hydroxy-5-(2-(2-(2-(2-((2Z,5Z,8Z,11Z,14Z,17Z)-eicosa-2,5,8,11,14,17-hexaenylthio)acetamido)ethyl)disulfanyl)acetamido)benzoate is dissolved in a mixture of THF and water with 5N NaOH. The mixture is stirred (50° C., 3 h) and the volatiles are removed under reduced pressure. The resulting aqueous mixture is extracted with EtOAc, and the combined organic extracts are washed with water, brine and dried over MgSO₄. Solvent evaporation affords 2-hydroxy-5-(2-(2-(2-(2-((2Z,5Z,8Z,11Z,14Z,17Z)-eicosa-2,5,8,11,14,17-hexaenylthio) acetamido)ethyl)disulfanyl)acetamido)benzoic acid.

### Example 3

### Preparation of 2-hydroxy-N-(2-(2-(2-((2Z,5Z,8Z,11Z,14Z,17Z)-eicosa-2,5,8,11,14,17-hexaenylthio)acetamido)ethoxy)ethyl)benzamide (1-1)

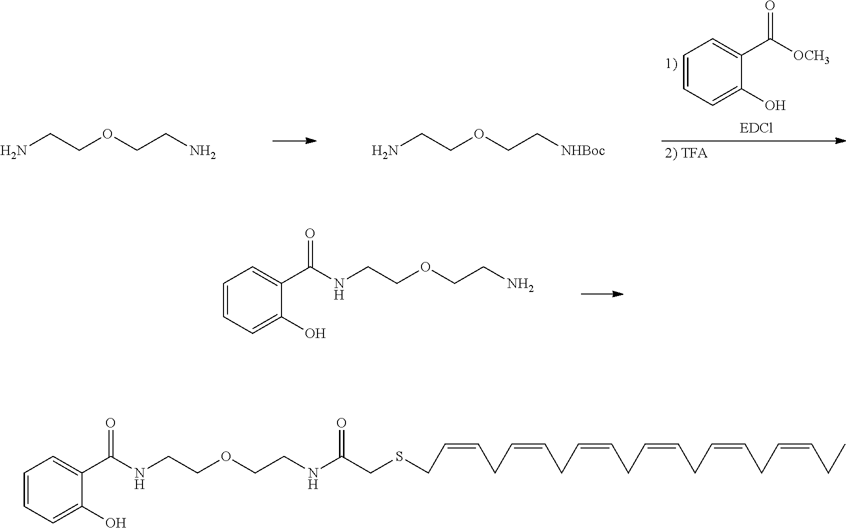

In a typical run, sodium hydroxide (400 mg, 10 mmol) is dissolved in MeOH (70 mL) and 2-(2-aminoethoxy)ethanamine dihydrochloride (1.0 g, 5.65 mmol) is added. The resulting reaction mixture is stirred at room temperature for 30 min. A solution containing Boc₂O (740 mg, 3.40 mmol) in THF (15 mL) is then added dropwise, at room temperature, over a period of 15 min. The resulting reaction mixture is stirred at room temperature for 18 h and then concentrated under reduced pressure. The resulting residue is taken up in CH₂Cl₂ (200 mL) and stirred vigorously at room temperature for 4 h. The mixture is filtered and the filtrate is concentrated under reduced pressure to afford tert-butyl 2-(2-aminoethoxy)ethylcarbamate (850 mg, 74%).

tert-Butyl 2-(2-aminoethoxy)ethylcarbamate (1.2 mmol) is then taken up in CH₃CN (10 mL) along with salicylic acid (1.2 mmol) and EDCI (1.5 mmol). The resulting reaction mixture is stirred at room temperature for 18 h. It is then diluted with EtOAc (20 mL), washed with saturated aqueous NaHCO₃, brine, dried over Na₂SO₄, filtered and concentrated under reduced pressure. The resulting residue is purified by silica gel chromatography (9:1 CH₂Cl₂/MeOH) to afford tert-butyl 2-(2-(2-hydroxybenzamido)ethoxy)ethylcarbamate.

tert-Butyl 2-(2-(2-hydroxybenzamido)ethoxy)ethylcarbamate (0.5 mmol) is taken up in 25% TFA in CH₂Cl₂ (5 mL) and allowed to stand at room temperature for 2 h. The resulting reaction mixture is concentrated under reduced pressure to afford the TFA salt of N-(2-(2-aminoethoxy)ethyl)-2-hydroxybenzamide. This material is taken up in CH₃CN (5 mL) along with 2-((2Z,5Z,8Z,11Z,14Z,17Z)-icosa-2,5,8,11,14,17-hexaenylthio)acetic acid (0.50 mmol), HATU (0.55 mmol) and DIEA (0.75 mmol). The resulting reaction mixture is stirred at room temperature for 2 h. It is then diluted with EtOAc and washed successively with saturated aqueous NaHCO₃ and brine. The organic layer is dried over Na₂SO₄, filtered and concentrated under reduced pressure. Purification by silica gel chromatography (5% MeOH—CH₂Cl₂) affords 2-hydroxy-N-(2-(2-(2-((2Z,5Z,8Z,11Z,14Z,17Z)-icosa-2,5,8,11,14,17-hexaenylthio)acetamido)ethoxy)ethyl)benzamide.

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

1. A molecular conjugate comprising a salicylate and a substituted thioacetic acid.

2. A compound of Formula I: 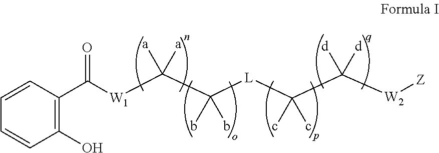
or a pharmaceutically acceptable salt, hydrate, solvate, prodrug, enantiomer, or stereoisomer thereof;
wherein
W₁ and W₂ are each independently null, O, S, NH, NR, or W₁ and W₂ can be taken together can form an imidazolidine or piperazine group;
each a, b, c, and d is independently —H, -D, —CH₃, alkyl —OCH₃, —OCH₂CH₃, —C(O)OR, —O—Z, or benzyl, or two of a, b, c, and d can be taken together, along with the single carbon to which they are bound, to form a cycloalkyl or heterocycle;
each n, o, p, and q is independently 0, 1, or 2;
each L is independently —O—, —S—, —S(O)—, —S(O)₂—, —S—S—, —(C₁-C₆alkyl)- 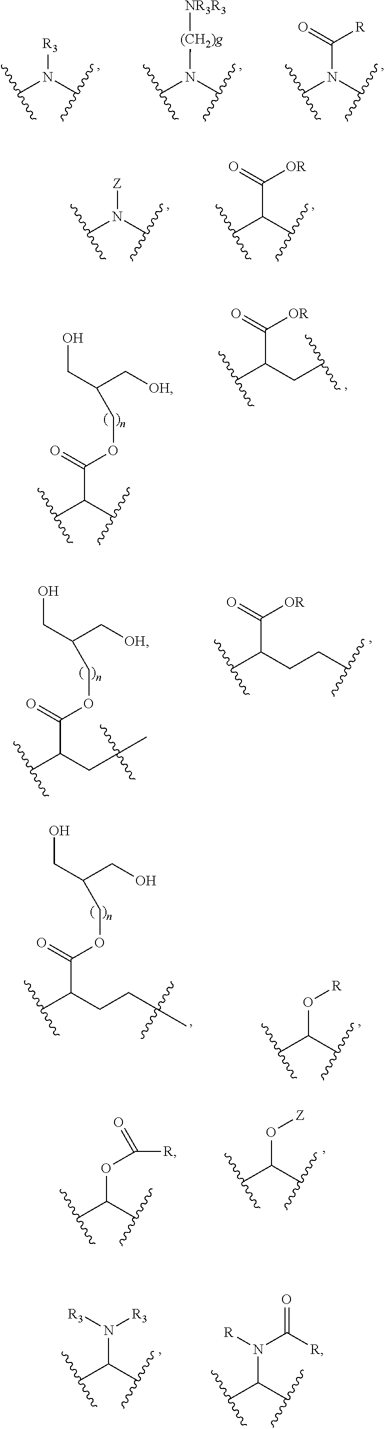 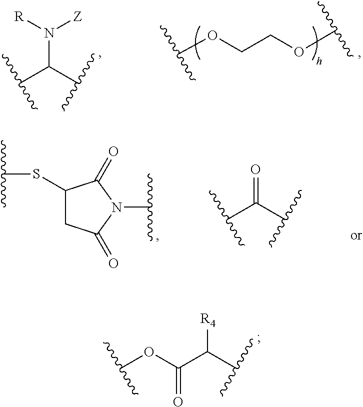
wherein the representation of L is not limited directionally left to right as is depicted, rather either the left side or the right side of L can be bound to the W₁ side of the compound of Formula I;
each g is independently 2, 3 or 4;
each h is independently 1, 2, 3 or 4;
m is 0, 1, 2, or 3; if m is more than 1, then L can be the same or different;
each R₃ is independently H or C₁-C₆ alkyl, or both R₃ groups, when taken together with the nitrogen to which they are attached, can form a heterocycle;
each R₄ is independently e, H or straight or branched C₁-C₁₀ alkyl which can be optionally substituted with OH, NH₂, CO₂R, CONH₂, phenyl, C₆H₄OH, imidazole or arginine;
each e is independently H or any one of the side chains of the naturally occurring amino acids;
each Z is independently —H, or 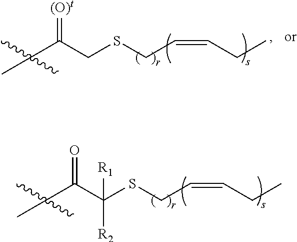
with the proviso that there is at least one 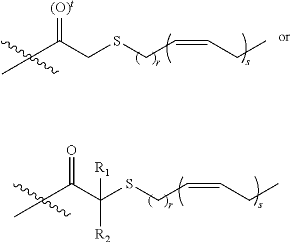
in the compound;
each r is independently 1, 2, 3, 10, 11, 12, 13, 14, 15, or 16,
each s is independently 0, 3, 4, 5, or 6;
with the proviso that if s is 0, then r is 10, 11, 12, 13, 14, 15, or 16;
each t is independently 0 or 1;
R₁ and R₂ are each independently hydrogen, deuterium, —C₁-C₄ alkyl, -halogen, —OH, —C(O)C₁-C₄ alkyl, —O-aryl, —O-benzyl, —OC(O)C₁-C₄ alkyl, —C₁-C₃ alkene, —C₁-C₃ alkyne, —C(O)C₁-C₄ alkyl, —NH₂, —NH(C₁-C₃ alkyl), —N(C₁-C₃ alkyl)₂, —NH(C(O)C₁-C₃ alkyl), —N(C(O)C₁-C₃ alkyl)₂, —SH, —S(C₁-C₃ alkyl), —S(O)C₁-C₃ alkyl, —S(O)₂C₁-C₃ alkyl; and
each R is independently —H, —C(O)—C₁-C₃ alkyl, or straight or branched C₁-C₄ alkyl optionally substituted with OR, NR₂, or halogen;
provided that
when each of m, n, o, p, and q, is 0, Wᵢ and W₂ are each null, and Z is 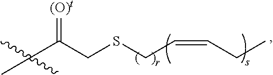
then t must be 0; and
when each of m, n, o, p, and q, is 0, and W₁ and W₂ are each null, then Z must not be

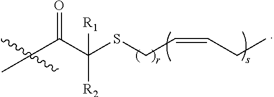

3. A compound of Formula II: 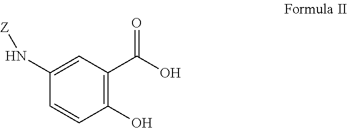
or a pharmaceutically acceptable salt, hydrate, solvate, prodrug, enantiomer, or stereoisomer thereof;
wherein
Z is 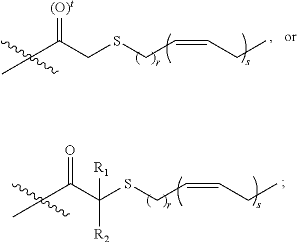
r is 1, 2, 3, 10, 11, 12, 13, 14, 15, or 16,
s is 0, 3, 4, 5, or 6;
with the proviso that if s is 0, then r is 10, 11, 12, 13, 14, 15, or 16;
t is 0 or 1; and
R₁ and R₂ are each independently hydrogen, deuterium, —C₁-C₄ alkyl, -halogen, —OH, —C(O)C₁-C₄ alkyl, —O-aryl, —O-benzyl, —OC(O)C₁-C₄ alkyl, —C₁-C₃ alkene, —C₁-C₃ alkyne, —C(O)C₁-C₄ alkyl, —NH₂, —NH(C₁-C₃ alkyl), —N(C₁-C₃ alkyl)₂, —NH(C(O)C₁-C₃ alkyl), —N(C(O)C₁-C₃ alkyl)₂, —SH, —S(C₁-C₃ alkyl), —S(O)C₁-C₃ alkyl, —S(O)₂C₁-C₃ alkyl.

4. A pharmaceutical composition comprising a molecular conjugate of claim 1 and a pharmaceutically acceptable carrier.

5. A pharmaceutical composition comprising a compound of claim 2 and a pharmaceutically acceptable carrier.

6. A pharmaceutical composition comprising a compound of claim 3 and a pharmaceutically acceptable carrier.

7. A method for treating a disease with inflammation as the underlying etiology comprising administering to a patient in need thereof an effective amount of a compound of claim 2.

8. The method of claim 7, wherein the disease with inflammation as the underlying etiology is selected from hypertriglyceridemia, hypercholesterolemia, fatty liver disease, atherosclerosis, coronary heart disease, Type 2 diabetes, diabetic nephropathy, diabetic neuropathy, diabetic retinopathy, metabolic syndrome, cardiovascular disease, or multiple sclerosis.

9. A method for treating a disease with inflammation as the underlying etiology, the method comprising administering to a patient in need thereof an effective amount of a compound of claim 3.

10. The method of claim 9, wherein the disease with inflammation as the underlying etiology is selected from hypertriglyceridemia, hypercholesterolemia, fatty liver disease, atherosclerosis, coronary heart disease, Type 2 diabetes, diabetic nephropathy, diabetic neuropathy, diabetic retinopathy, metabolic syndrome, cardiovascular disease, or multiple sclerosis.
